(19) United States
(12) Patent Application Publication
(10) Pub. No.: US 2021/0375159 A1
(43) Pub. Date: Dec. 2, 2021

(54) Kit and Method for Home Use for Interproximal Reduction (IPR) Stripping of Teeth for Braces or Aligners

(71) Applicant: Marc Lemchen, New York, NY (US)

(72) Inventor: Marc Lemchen, New York, NY (US)

(21) Appl. No.: 17/320,644

(22) Filed: May 14, 2021

Related U.S. Application Data
(60) Provisional application No. 63/033,623, filed on Jun. 2, 2020.

(51) Int. Cl. G09B 23/28; A61C 3/025; G09B 5/04; G09B 5/02; A61C 7/00

(52) U.S. Cl. CPC G09B 23/283; A61C 3/025; A61C 7/002; G09B 5/02; G09B 5/04

(57) ABSTRACT

An interproximal reduction (IPR) kit and method for using the same. The patient takes their own dental impression and submits it to a dental service provider for a treatment plan. A set of aligners based on the treatment plan are then delivered to the patient as part of a kit. The kit also includes a set of written instructions and/or a treatment diagram and a plurality of strips used to reduce the widths of specific spots of the patient's teeth according to the treatment plan. The patient follows the reapproximation instructions or is instructed to go to a website for supervision for the performance of the reapproximation process. After sufficient instruction, the patient applies the plurality of strips to the correct spots between their teeth. Once reapproximation is complete, the patient is ready to begin wearing the aligners as dictated by the treatment plan.

This application claims priority to, and the benefit of the earlier filing date of U.S. provisional patent application entitled “Kit and Method for Home Use for Interproximal Reduction (IPR) Stripping of Teeth for Braces or Aligners,” filed on Jun. 2, 2020, Ser. No. 63/033,623, pursuant to 35 USC 119, the contents of all of which are incorporated herein by reference.

### BACKGROUND

### Field of the Technology

The invention relates to the field of orthodontic devices and methods, specifically to interproximal reduction of a patient's teeth.

### Description of the Prior Art

In orthodonture, teeth are moved or realigned in the jawline by gentle persistence forces applied to the teeth in a directed manner through the use of orthodontic appliances, usually through braces or a series of aligners.

In order for teeth to move or rotate, there must be space in the jawline for the movement or rotation to occur. This requires there to be a predefined amount of selected open space between the teeth in the appropriate locations to allow the selected tooth or teeth to be moved or rotated.

In some occasions the movement is so large that the orthodontist needs to extract a tooth, or expand the dental arches, to provide the space in a jawline that is too crowded. However, in most cases, the space between the teeth is sufficient to allow the needed movement or rotation. Where such space is not naturally occurring, it must be artificially created, which is done by the orthodontist using an interproximal polishing disk, a rotating burr, or a sanding strip to selectively remove a small amount of enamel from the tooth to be moved or one or both adjacent teeth. Often the amount of clearance or space that needs to be created is as little of 0.1 mm or less but could be as much as 0.5 mm.

Given the availability of internet videos for patient instruction and training, and the wide availability of digital, internet compatible cameras in cellphones, it is increasingly the case that an orthodontic patient can undertake more self-help procedures in his or her orthodontic care as interactively guided by his or her orthodontist.

Reapproximation is also used to reduce what are called “black triangles” which are formed when the recession of the gum tissue, particularly on rotated teeth, will result in a “gap” at the gum line, all teeth being wider at their incisal or occlusal edge than where the root begins and the crown ends.

What is needed is kind of kit for home use for interproximal reduction (IPR) stripping of teeth for braces or aligners as well as a do-it-yourself workflow or methodology to be used in conjunction with a do-it-yourself aligner therapy to allow more significant crowding and alignment issues to be addressed without the patient/client needing to be seen by a dental professional requiring travel, exposure to any health challenges, and additional costs.

### BRIEF SUMMARY

The illustrated embodiments include an improvement in an orthodontic or dental work flow including a step of facilitating the patient's providing their own “reapproximation” as prescribed in his or her treatment plan using devices, tools, instruments, provided with his or her aligners at home. The patient's self-work is supported with instructions, which are written and/or in video form and, if required, a supervised online interactive video during the reapproximation process. The initial “instruments” provided include abrasive dental finishing strips of varying thicknesses and abrasiveness, which may be color coded or clearly marked, a diagram indicating where to do the reapproximation, and which instrument to use. This instrument could be just an abrasive strip, a diamond strip, or a strip in a conventional handle type of holder or jig.

The patient takes his or her own impression (or may go to a scanning location, either in a professional office or a free standing location not affiliated with a licensed professional directly), submits the impression for a treatment plan which is then developed and approved by a licensed individual. The patient approves the treatment plan and aligners are manufactured and delivered to the patient. The patient places the aligners and follows provided reapproximation instructions or is instructed to go to a site for supervision of the performance of the reapproximation process. The tele-video instruction may optionally be set by appointment or may be a “walk-in” or demand online pick up and connect.

The invention provides a kit for interproximal reduction (IPR) stripping of teeth for braces or aligners by a patient at home. The kit includes a plurality of sanding strips and an instructional means which trains or teaches the patient how to selectively use the plurality of sanding strips to establish individually determined IPR of their own teeth. Each of the sanding strips are of a different thickness, and each of the sanding strips is further keyed or dedicated for insertion and use in at least one of a number of different predetermined positions between of the patient's teeth.

In one particular embodiment, the kit also includes a treatment plan, the treatment plan itself including a graphical representation of the patient's teeth and a set of markers that are disposed within the graphical representation. Each one of the markers denotes or represents a corresponding one of the predetermined positions between of the patient's teeth. Furthermore, each of the sanding strips is keyed or matched to at least one of the plurality of markers.

In another embodiment, each of the sanding strips within the kit has a different degree of abrasiveness relative to each other.

In a further embodiment, the instructional means to train the patient how to selectively use the sanding strips within the kit is an audio or visual recording which instructs the patient how to use the plurality of sanding strips when performing an IPR. The audio or visual recording instructing the patient how to use the sanding strips is transmitted to the patient through a network which connects the patient to a dental service provider.

In an alternative embodiment, the instructional means to train the patient how to selectively use the sanding strips within the kit is a set of written instructions. Here, the set of written instructions may be disposed on or incorporated into the treatment plan.

The invention further provides a method for using a kit at home for interproximal reduction (IPR) stripping of teeth for braces or aligners by a patient. The method includes providing the patient with a number of sanding strips that each comprise a different relative thickness and then training the patient how to selectively use the sanding strips in order to establish the individually determined IPR of the patient's teeth according to the predetermined treatment plan. The method further includes selectively using at least one of the sanding strips to create at least one interproximal space in at least one of a number of positions between selected teeth of the patient according to the predetermined treatment plan.

In one embodiment, the method further includes providing the patient with a reference containing the predetermined treatment plan. In this embodiment, the reference includes a graphical representation of the patient's teeth and multiple markers disposed within the graphical representation of the patient's teeth. Additionally, each one of the markers denotes a corresponding one of the positions between selected teeth of the patient where at least one interproximal space is to be created, while each of the sanding strips is keyed or matched to at least one of the markers.

In a further embodiment, providing the patient with sanding strips that each have a different relative thickness further includes providing the patient with multiple sanding strips that each comprise a different relative abrasiveness or grit.

In yet another embodiment, training the patient how to selectively use the sanding strips according to the predetermined treatment plan specifically includes training the patient how to selectively use the sanding strips in order to establish individually determined IPR of the patient's teeth according to the predetermined treatment plan remotely by video and/or teledentistry by a live dental service provider.

In an alternative embodiment, training the patient how to selectively use the sanding strips according to the predetermined treatment plan specifically includes providing the patient with an audio or visual recording which trains the patient how to selectively use the sanding strips in order to establish individually determined IPR of the patient's teeth according to the predetermined treatment plan. Here, the audio or visual recording may be transmitted to the patient through a network which connects the patient to a dental service provider.

In yet another embodiment, the method further includes additionally training the patient how to selectively use the sanding strips to establish individually determined IPR of the patient's teeth according to the predetermined treatment plan if and when the initial selective use of any of the sanding strips to create at least one interproximal space in at least one of a plurality of positions between selected teeth of the patient according to the predetermined treatment plan is determined to be unsuccessful by a dental service provider.

The invention further provides an improvement in a method for using a kit for interproximal reduction (IPR) stripping of teeth for braces or aligners by a patient at home for repositioning one or more teeth of the patient. The improvement includes acquiring a representation of the spatial alignment of the patient's teeth and then producing a treatment plan for the patient based on the representation of the spatial alignment of the patient's teeth by a dental or orthodontic professional, the treatment plan itself containing an individually determined IPR of the patient's teeth. Next, multiple sanding strips are sent to the patient, wherein each of the sanding strips are configured to establish individually determined IPR of the patient's teeth. The patient is then instructed how to selectively use the sanding strips in order to establish individually determined IPR of their teeth according to the treatment plan. After being instructed, the patient then selectively uses at least one of the sanding strips to create at least one interproximal space between selected teeth in order to establish individually determined IPR of the their teeth according to the treatment plan. Here, each of the sanding strips are keyed or matched for insertion and use in at least one of multiple positions between selected teeth of the patient.

In one embodiment, acquiring a representation of the spatial alignment of the patient's teeth specifically includes performing an intraoral scan of the patient's teeth, creating a dental impression of the patient's teeth, or performing a photographic scan of the patient's teeth.

In yet another embodiment, instructing the patient how to selectively use the sanding strips in order to establish individually determined IPR of their teeth according to the treatment plan specifically includes training the patient remotely through video and/or teledentistry.

In a further embodiment, the method also includes transmitting to the patient an indication of an approval of the treatment plan by the dental or orthodontic professional, wherein the approval is received without the dental or orthodontic professional having physically been proximate to or physically seen the patient;

In a related embodiment, the method also includes manufacturing at least one aligner based on the treatment plan, the at least one aligner customized to the patient and configured to reposition one or more teeth of the patient in accordance with the treatment plan wherein the user receives orthodontic treatment without ever having physically seen a dental or orthodontic professional.

In a further embodiment, instructing the patient how to selectively use the sanding strips in order to establish individually determined IPR of their teeth according to the treatment plan specifically includes sending the patient with a reference containing the treatment plan. Here, the reference includes a graphical representation of the patient's teeth and multiple markers that are disposed within the graphical representation. Furthermore, each one of the markers denotes or is matched with at least one of multiple positions between selected teeth of the patient where at least one interproximal space is to be created, each of the sanding strips in turn being keyed or matched to at least one of the markers.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The disclosure can be better visualized by turning now to the following drawings wherein like elements are referenced by like numerals.

### BRIEF DESCRIPTION OF THE DRAWINGS

FIG. 1 is a depiction of a patient's teeth with a black triangle between two adjacent incisors.

FIG. 2 is the depiction of FIG. 1 where a polishing or sanding strip in inserted between the incisors of FIG. 1 and used to reduce the enamel of both teeth at their closest proximity.

FIG. 3 is the depiction of FIG. 2 where an orthodontic appliance or braces have been fitted to the teeth, although equivalently a series of aligners could have been provided and fitted.

FIG. 4 is the depiction of FIG. 3 after the orthodontic appliance has closed the gap between the IPR treated incisors and the removed.

FIG. 5 is a flowchart of the implementation of one embodiment, of the method of the invention.

FIG. 6 is a depiction of an IPR treatment plan illustrative of what may be given to a patient.

FIG. 7 is a perspective view of a plurality of polishing or sanding strips comprising a handle which may be given to a patient.

The disclosure and its various embodiments can now be better understood by turning to the following detailed description of the preferred embodiments which are presented as illustrated examples of the embodiments defined in the claims. It is expressly understood that the embodiments as defined by the claims may be broader than the illustrated embodiments described below.

### DETAILED DESCRIPTION OF THE PREFERRED EMBODIMENTS

The illustrated embodiments of the current invention include an improvement in a method for using a kit for home use for interproximal reduction (IPR) stripping of teeth for braces or aligners by a patient for repositioning one or more teeth of a user. The method includes that claimed and disclosed in U.S. Pat. No. 10,636,522, incorporated herein by reference in its entirety. In that method a patient: receives, by an appointment management system, a request to schedule an appointment at an intraoral scanning site, the intraoral scanning site having an intraoral scanner configured to scan a mouth of a user, the appointment being for a technician to conduct an intraoral scan of the mouth of the user at the intraoral scanning site without a dentist or orthodontist physically seeing the user during the scheduled appointment, wherein the technician is not a dentist or an orthodontist; schedules, through the appointment management system, the appointment at the intraoral scanning site in accordance with the request; receives from the appointment management system, a message to a device of the patient, the message including a confirmation confirming the scheduled appointment; is scanned by the intraoral scanner during the scheduled appointment, the intraoral scan generating three-dimensional data of the mouth of the patient; receives from a treatment plan computing system located at a treatment plan site, a treatment plan for the patient based on the three-dimensional data of the mouth of the patient; receives an indication of an approval of the treatment plan by a dental or orthodontic professional, wherein the approval is received without the dental or orthodontic professional having physically seen the patient; and is provided, from a fabrication site, a plurality of aligners based on the treatment plan, the plurality of aligners specific to the patient and being configured to reposition one or more teeth of the user in accordance with the treatment plan wherein the user receives orthodontic treatment without ever having physically seen the approving dental or orthodontic professional.

The improvement in the method includes the steps of training the patient at the intraoral scanning site, remotely by video and/or teledentistry how to selectively use the plurality of polishing strips to establish individually determined IPR of the patient's teeth according to a predetermined treatment plan or workflow provided from the treatment plan site, and selectively using ones of a plurality of polishing or sanding strips of correspondingly different thicknesses to create interproximal spaces between selected teeth of the patient according to a predetermined treatment plan or workflow from the treatment plan site, which strips are keyed for insertion and use between selected teeth.

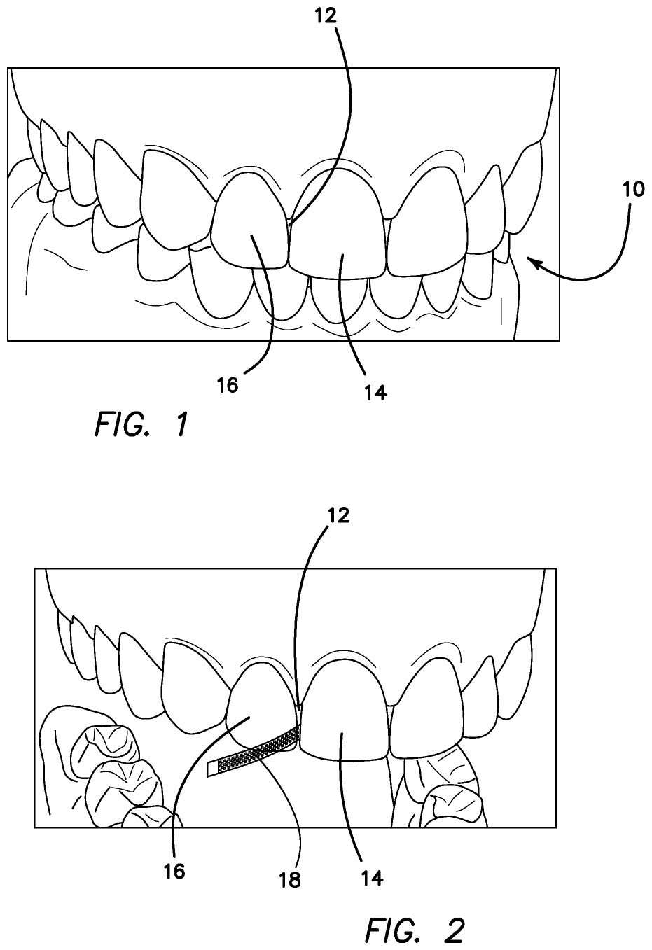

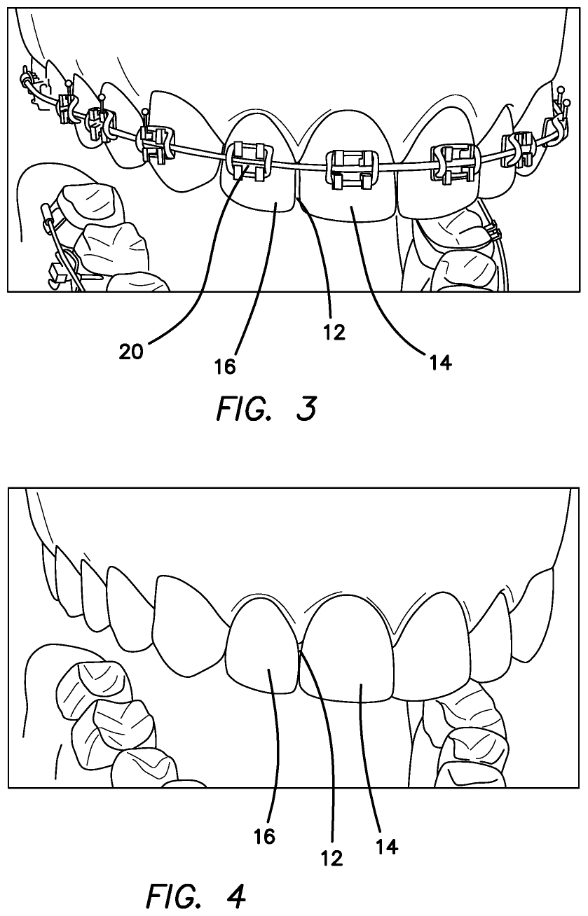

Turning to FIG. 1, a depiction of a patient's teeth 10 with a black triangle or gap 12 between two adjacent incisors 14 and 16 may be seen. FIG. 2 is the depiction of FIG. 1 where a selected polishing or sanding strip 18 in inserted by the patient between the incisors 14, 16, pursuant to the remote or distance training and treatment plan of the illustrated embodiments of the invention, and used to reduce the enamel of both teeth at their closest proximity. A black triangle 12 can occur between any two adjacent teeth 10 and incisors 14 and 16 have been shown only for the purpose of illustration. As discussed below, the IPR treatment plan is integrated into a more comprehensive dental imaging or modeling procedure from which an orthodontic procedure, such as IPR, can be combined. FIG. 3 is the depiction of FIG. 2 where an orthodontic appliance or braces 20 have been fitted to the teeth after performing the IPR treatment plan, although equivalently a series of aligners could have been provided and fitted. FIG. 4 is the depiction of FIG. 3 after the orthodontic appliance 20 has been employed according to orthodontic treatment to close the gap 12 between the IPR treated incisors 14, 16.

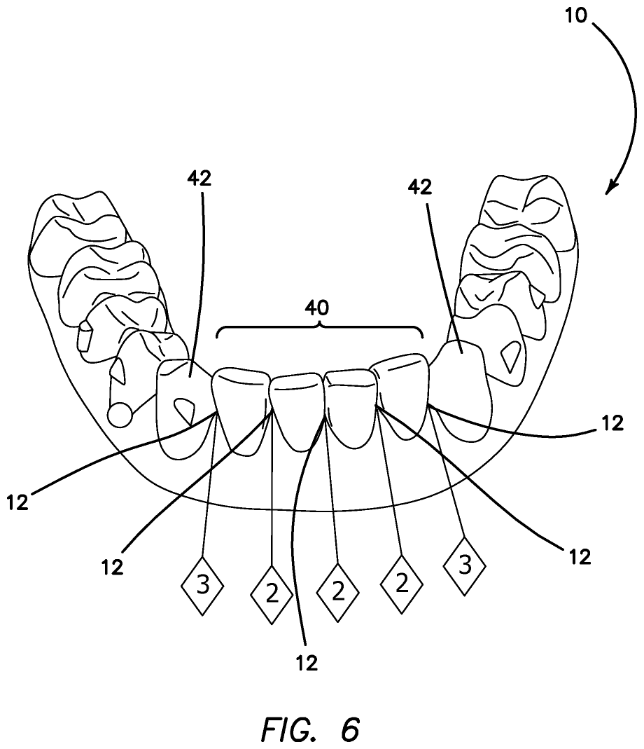

FIG. 6 is a depiction of an IPR treatment diagram illustrative of what may be given to a patient in order to facilitate the treatment plan as determined by the dental services professional. What is depicted is the lower teeth 10 with five gaps 12 between the front lower incisors 40 and adjacent canines 42. Further indicated in the diagram of FIG. 6 is that a number keyed strip 18, in the illustration identified by the markers “2” or “3”, is to be applied in each of the gaps 12. Each gap 12 will be sanded or polished until the narrowest space between the teeth 40, 42 is increased to an amount that is determined by the thickness of the appropriately keyed strip 18. In the illustrated embodiment of FIG. 6, the strip 18 is identical in each of the incisor-incisor gaps 12 (keyed as #2 strip 18) but has a different degree of abrasiveness and/or thickness in each of the incisor-canine gaps 12 (keyed as #3 strip 18). Once the IPR process has been completed, the patient's teeth are now ready for an orthodontic treatment such as a series of aligners to be applied to the teeth 10 in order to close the widened tooth separations created by the patient-performed IPR.

It should be noted however that the strip 18 comprising two different levels of abrasiveness or thicknesses is for illustrative purposes only and that the relative abrasiveness and/or thickness of each strip 18 may be selected according to the approved treatment plan. Additionally, instead of being keyed to a numeral marker such as “#2” or “#3”, each strip 18 may have its corresponding value or thickness keyed to a specific color, symbol, or other graphical image. For example, for the strips 18 to be used between adjacent incisors 40, each strip may be keyed to denote a first thickness by a blue coloring or marking, while each strip 18 to be used between an incisor 40 and a canine 42 may be keyed to denote a second thickness by a red coloring or marking.

The illustrated embodiments of the method include the step of providing a patient with a plurality of sanding strips 18 in a variety of sizes or thicknesses appropriate for each interproximal location determined as a treatment site for that patient. The patient, trained by written instructions, video instructions, on-line supervised sessions during which the patient is guided through the procedure, or his or her orthodontist, places the strip 18 between the selected teeth and gently reciprocates the strip 18 to sand down one or both sides of the closest enamel portions of the selected adjacent teeth, usually at or near the crowns of the teeth without pressing the sanding strip 18 to one side or the other. The sanding strip 18 may have a polishing or finely abrasive surface on one or both of its sides as determined by the needed treatment. When the desired clearance is created, the sanding strip 18 will no longer frictionally drag across the tooth to any substantial degree.

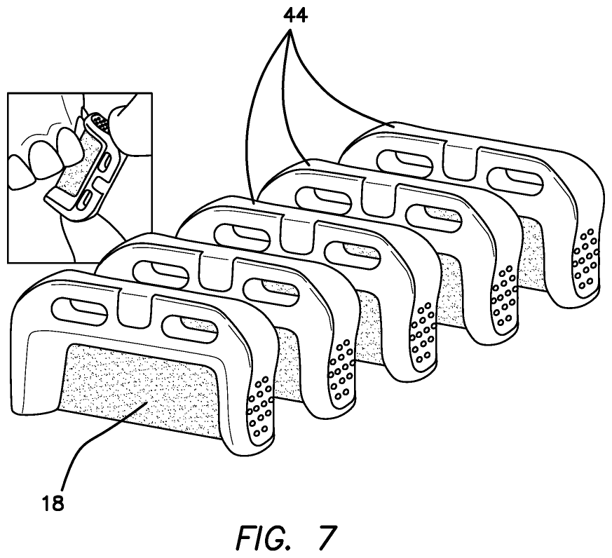

In an alternative embodiment, each sanding strip 18 may be placed or inserted into a tool or jig or may otherwise comprise a handle which allows the user to maintain a better grip on the sanding strip 18 and thus maintain better control over the sanding strip 18 as it is used to polish or refine the surface of the patient's teeth. A plurality of handles 44 may be provided to the patient as part of the kit as seen in FIG. 7, with each one of the plurality of handles 44 comprising a different color, symbol, character, or numeral value disposed thereon which is keyed to a specific sanding strip 18 comprising a predetermined thickness and/or abrasiveness. In other words, the patient may visually confirm the thickness and/or abrasiveness a particular sanding strip 18 by looking at the handle 44 accommodating or holding the sanding strip 18. The handles 44 are preferably provided to the patient with each corresponding or keyed sanding strip 18 coupled to it or maintained therein, however in one particular embodiment, the patient is provided with only a single handle 44 and may instead change or swap out the different sanding strips 18 as needed in order to complete their predetermined treatment plan. Each sanding strip 18 may be permanently coupled to a corresponding handle 44, however in a preferred embodiment, each sanding strip 18 may be selectively removed from a corresponding handle 44 should replacement of the sanding strip 18 be necessary or if any of the handles 44 need to be stored for later use or disposed of.

The patient is provided with a diagram illustrating between which teeth and how much space to create between the teeth and if the “sanding” process should be applied to both adjacent teeth or to only one or the other where the spaced is to be created. The patient is trained remotely by video or in the orthodontist's office to loosely hold the sanding strip 18 between the teeth without pressing or bending it towards either side. Alternatively, the handle 44 currently holding the sanding strip 18, while it is being manually or automatically reciprocated, is designed to apply little if any side or lateral force against the adjacent teeth. The force brought to bear by the sanding strip 18 against the sides of the teeth arises substantially only by reason of the predetermined thickness of the strip according to the determined treatment.

For example, if a 0.2 mm space between the lower central incisors, and 0.3 mm space between the lower left central and lateral incisors is called for by the treatment, two different sanding strips 18 are provided to the patient. One strip 18 has a 0.2 mm thickness, and while the other has a 0.3 mm thickness. The patient is provided with instructions, a video or other training media, or a teledentistry visit is performed to allow the patient to perform this all-important IPR step in aligner therapy, which is common to most aligner treatments.

These procedures could also be part of the workflow not involving a scan but initiating with the client/patient receiving an “impression kit” at home, being instructed in taking their own impression, and that impression submitted to design and create the series of aligners to align the teeth and or modify the bite.

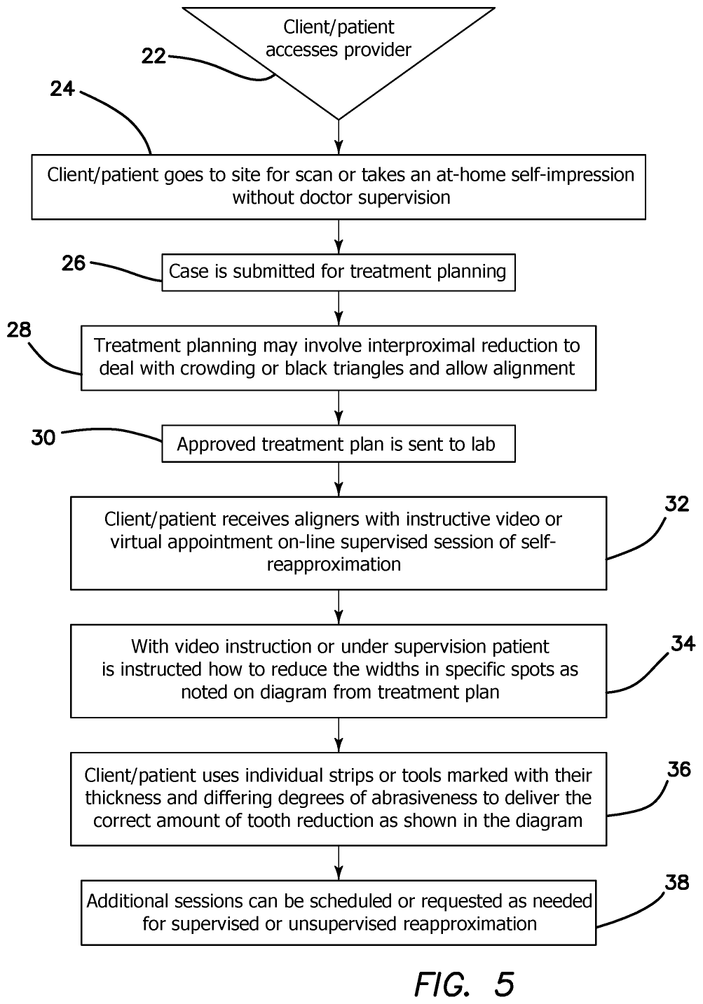

FIG. 5 is a flowchart of the implementation of one embodiment of the illustrated invention. It is to be understood that that methodology of FIG. 5 can be incorporated into any other automated teledentistry or computer assisted dental service procedure or method. In step 22 the client or patient accesses the dental services provider via the internet or other remote means. Specifically, the client/patient goes to an internet service site for an intraoral scan of his or her teeth, for example, using his or her cellphone camera, or takes an at-home self-impression without doctor supervision at step 24 using a do-it-yourself kit and instructions from the dental service provider. The impression is mailed and/or the scan is transmitted to the dental provider and is submitted for planning of an orthodontic treatment at step 26. Treatment planning at step 28 may be determined to require interproximal reduction (IPR) to address crowding or black triangles and to allow alignment. The dental service provider sends the approved treatment plan to an orthodontic lab at step 30 where a series of aligners, which will implement the plan is manufactured and are sent to the patient. The patient receives the aligners at step 32 with an instructive video or a virtual appointment for on-line supervised session of self-reapproximation (IPR) under the treatment plan. At step 34 the video instruction or using video supervision is used to instruct the patient how to reduce the widths in specific spots as noted on a diagram from the treatment plan. At step 36 the patient uses individual strips 18 or tools marked with their thickness and differing degrees of abrasiveness to deliver the correct amount of tooth reduction as shown in the treatment diagram. Additional remote sessions can be scheduled or requested from the dental service provider as needed for additional supervised or unsupervised reapproximation using teledenistry procedures at step 38.

Many alterations and modifications may be made by those having ordinary skill in the art without departing from the spirit and scope of the embodiments. Therefore, it must be understood that the illustrated embodiment has been set forth only for the purposes of example and that it should not be taken as limiting the embodiments as defined by the following embodiments and its various embodiments.

Therefore, it must be understood that the illustrated embodiment has been set forth only for the purposes of example and that it should not be taken as limiting the embodiments as defined by the following claims. For example, notwithstanding the fact that the elements of a claim are set forth below in a certain combination, it must be expressly understood that the embodiments includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the embodiments is explicitly contemplated as within the scope of the embodiments.

The words used in this specification to describe the various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the embodiments.

I claim:

1. A kit for home use for interproximal reduction (IPR) stripping of teeth for braces or aligners by a patient comprising:
a plurality of sanding strips; and
an instructional means to train the patient how to selectively use the plurality of sanding strips to establish individually determined IPR of the patient's teeth,
wherein each of the plurality of sanding strips comprises a different thickness, and
wherein each of the plurality of sanding strips is keyed for insertion and use in at least one of a plurality of predetermined positions between of the patient's teeth.

2. The kit of claim 1 further comprising a treatment plan, the treatment plan comprising:
a graphical representation of the patient's teeth; and
a plurality of markers disposed within the graphical representation of the patient's teeth,
wherein each one of the plurality of markers denotes a corresponding one of the plurality of predetermined positions between of the patient's teeth, and
wherein each of the plurality of sanding strips is keyed to at least one of the plurality of markers.

3. The kit of claim 1 wherein each of the plurality of sanding strips comprises a different degree of abrasiveness relative to each other.

4. The kit of claim 1 wherein the instructional means to train the patient how to selectively use the plurality of sanding strips to establish individually determined IPR of the patient's teeth comprises an audio or visual recording instructing the patient how to use the plurality of sanding strips when performing an IPR.

5. The kit of claim 1 wherein the instructional means to train the patient how to selectively use the plurality of sanding strips to establish individually determined IPR of the patient's teeth comprises a set of written instructions.

6. The kit of claim 5 wherein the instructional means to train the patient how to selectively use the plurality of sanding strips to establish individually determined IPR of the patient's teeth comprises a set of written instructions disposed on the treatment plan.

7. The kit of claim 4 wherein the audio or visual recording instructing the patient how to use the plurality of sanding strips when performing an IPR is transmitted to the patient through a network connecting the patient to a dental service provider.

8. A method for using a kit for home use for interproximal reduction (IPR) stripping of teeth for braces or aligners by a patient comprising the steps of:
providing the patient with a plurality of sanding strips that each comprise a different relative thickness;
training the patient how to selectively use the plurality of sanding strips to establish individually determined IPR of the patient's teeth according to the predetermined treatment plan; and
selectively using at least one of the plurality of sanding strips to create at least one interproximal space in at least one of a plurality of positions between selected teeth of the patient according to the predetermined treatment plan.

9. The method of claim 8 further comprising providing the patient with a reference containing the predetermined treatment plan, the reference comprising:
a graphical representation of the patient's teeth; and
a plurality of markers disposed within the graphical representation of the patient's teeth,
wherein each one of the plurality of markers denotes a corresponding one of the plurality of positions between selected teeth of the patient where at least one interproximal space is to be created, and
wherein each of the plurality of sanding strips is keyed to at least one of the plurality of markers.

10. The method of claim 8 where providing the patient with a plurality of sanding strips that each comprise a different relative thickness comprises providing the patient with a plurality of sanding strips that each comprise a different relative abrasiveness.

11. The method of claim 8 where training the patient how to selectively use the plurality of sanding strips to establish individually determined IPR of the patient's teeth according to the predetermined treatment plan comprises training the patient how to selectively use the plurality of sanding strips to establish individually determined IPR of the patient's teeth according to the predetermined treatment plan remotely by video and/or teledentistry by a dental service provider.

12. The method of claim 8 where training the patient how to selectively use the plurality of sanding strips to establish individually determined IPR of the patient's teeth according to the predetermined treatment plan comprises providing the patient with an audio or visual recording training the patient how to selectively use the plurality of sanding strips to establish individually determined IPR of the patient's teeth according to the predetermined treatment plan.

13. The method of claim 12 where providing the patient with an audio or visual recording training the patient how to selectively use the plurality of sanding strips to establish individually determined IPR of the patient's teeth according to the predetermined treatment plan comprises transmitting the audio or visual recording to the patient through a network connecting the patient to a dental service provider.

14. The method of claim 8 further comprising additionally training the patient how to selectively use the plurality of sanding strips to establish individually determined IPR of the patient's teeth according to the predetermined treatment plan if the method step of selectively using at least one of the plurality of sanding strips to create at least one interproximal space in the at least one of a plurality of positions between selected teeth of the patient according to the predetermined treatment plan is determined to be unsuccessful by a dental service provider.

15. An improvement in a method for using a kit for interproximal reduction (IPR) stripping of teeth for braces or aligners by a patient at home for repositioning one or more teeth of the patient, the method comprising:
acquiring a representation of the spatial alignment of the patient's teeth;
producing a treatment plan for the patient based on the representation of the spatial alignment of the patient's teeth by a dental or orthodontic professional, wherein the treatment plan comprises establishing individually determined IPR of the patient's teeth;
sending a plurality of sanding strips to the patient, wherein each of the plurality of sanding strips are configured to establish individually determined IPR of the patient's teeth;
instructing the patient how to selectively use the plurality of sanding strips to establish individually determined IPR of the patient's teeth according to the treatment plan; and
selectively using at least one of the plurality of sanding strips to create at least one interproximal space between selected teeth of the patient to establish individually determined IPR of the patient's teeth according to the treatment plan, wherein each of the plurality of sanding strips are keyed for insertion and use in at least one of a plurality of positions between selected teeth of the patient.

16. The method of claim 15 where acquiring a representation of the spatial alignment of the patient's teeth comprises performing an intraoral scan of the patient's teeth, creating a dental impression of the patient's teeth, or performing a photographic scan of the patient's teeth.

17. The method of claim 15 where instructing the patient how to selectively use the plurality of sanding strips to establish individually determined IPR of the patient's teeth according to the treatment plan comprises training the patient remotely through video and/or teledentistry.

18. The method of claim 15 further comprising transmitting to the patient an indication of an approval of the treatment plan by the dental or orthodontic professional, wherein the approval is received without the dental or orthodontic professional having physically been proximate to or physically seen the patient;

19. The method of claim 15 further comprising manufacturing at least one aligner based on the treatment plan, the at least one aligner customized to the patient and configured to reposition one or more teeth of the patient in accordance with the treatment plan wherein the user receives orthodontic treatment without ever having physically seen a dental or orthodontic professional.

20. The method of claim 15 where instructing the patient how to selectively use the plurality of sanding strips to establish individually determined IPR of the patient's teeth according to the treatment plan comprises sending the patient with a reference containing the treatment plan, the reference comprising:
a graphical representation of the patient's teeth; and
a plurality of markers disposed within the graphical representation of the patient's teeth,
wherein each one of the plurality of markers denotes a corresponding one of a plurality of positions between selected teeth of the patient where at least one interproximal space is to be created, and
wherein each of the plurality of sanding strips is keyed to at least one of the plurality of markers.
